(19) United States
(12) Patent Application Publication
(10) Pub. No.: US 2024/0410886 A1
(43) Pub. Date: Dec. 12, 2024

(54) IMMUNOASSAYS

(71) Applicant: NABAS AS, Ås (NO)

(72) Inventor: Andrew CAMPBELL, Ås (NO)

(21) Appl. No.: 18/697,274

(22) PCT Filed: Sep. 30, 2022
(86) PCT No.: PCT/EP2022/077389

§ 371 Date: Mar. 29, 2024

(30) Foreign Application Priority Data
Sep. 30, 2021 (GB) 2114018.1

(51) Int. Cl. G01N 33/563; G01N 33/543

(52) U.S. Cl. CPC G01N 33/563; G01N 33/54346

(57) ABSTRACT

The present invention relates to immunoassays for identifying one or more types of free light chains. In particular the invention relates to a method for assaying Free Light Chains (FLCs) in a mammalian sample comprising the use of at least one avian-derived antibody (IgY). The invention also relates to a method for screening, diagnosis, monitoring or prognosis of a disease in a patient comprising conducting the assay method. The invention also relates to the use of avian-derived antibodies (IgY) in an immunoassay for assaying FLCs in a mammalian sample.

### FIELD OF THE INVENTION

The present invention relates to immunoassays for identifying one or more types of free light chains.

### BACKGROUND OF THE INVENTION

Antibodies are composed of two identical heavy chains and two identical light chains, each containing variable and constant domains. There are two types of light chains: kappa (κ) and lambda (λ). Each antibody expresses only one class of light chain. Usually, the light chains are attached to the heavy chains, and are then called bound light chains. However, there is normally some excess light chains that do not combine with heavy chains, these are called “free light chains” (FLCs). FLCs are detectable, for example, in the serum, plasma or urine of individuals.

Serum free light chains (sFLCs) are important biomarkers for the diagnosis and management of immune-related diseases such as smouldering multiple myeloma (SMM), multiple myeloma (MM), and other plasma cell disorders, such as monoclonal gammopathy of undetermined significance (MGUS), light-chain amyloidosis (AL-amyloidosis), and light-chain deposition disease (LCDD). They are also markers of immune-stimulating diseases, e.g. multiple sclerosis, some liver diseases and autoimmune diseases, such as systemic lupus erythematosus (SLE).

Normal and abnormal plasma cells produce more light chains than heavy chains, and these excess light chains are released into the bloodstream. These FLCs are typically cleared rapidly and metabolised by the kidneys. Increases in sFLCs may occur due to decreased renal clearance, increased polyclonal immunoglobulin production or monoclonal gammopathies.

FLCs may be detected by raising antibodies against the surface of the FLCs that, in the whole molecule immunoglobulin, is normally hidden by the binding of the light chain to the heavy chain. Methods for raising mammalian antibodies to FLCs have been previously disclosed (Bradwell et al, Clin. Chem., (2001), 47, 673-680).

Immunoassays are based on measuring the reaction between an antigen (e.g. a site on a protein) and an antibody specific to that antigen. Detection of free light chains can be carried out by immunoassays where antibodies are raised against at least one antigen in the region of the FLC which binds to the heavy chain in the complete antibody. There are commercially available kits for the detection of κ or λ FLCs, for example, “Freelite™”, manufactured by The Binding Site Limited and “N Latex” manufactured by Siemens. These are based on polyclonal sheep antibody technology and multiple monoclonal antibodies, respectively.

A problem with current sFLC assays is that they are not interchangeable, as different assay methods may provide different results on the same sample. One problem reported for assays for FLCs is that these molecules are known to be a heterogeneous group, demonstrating considerable variation, and often existing in polymeric forms in blood, serum, plasma, and urine. These may be, for example, dimers, tetramers, or higher polymeric forms. The κ FLCs are usually found as monomers, whereas the λ FLCs tend to form dimers (Sölling, Scand. J. Clin. Lab. Invest., (1976), 36, 447-452). Unless anti-FLC antibodies recognise all molecular forms in an equimolar manner, sFLC assays will not give equivalent results in all samples. Caponi et al (Clin. Chem. Lab. Med., (2016), 54, 1111-1113) highlighted this discrepancy between existing FLC assays.

The monomer/dimer ratio of serum FLC may also be different in healthy individuals versus patients with a monoclonal gammopathy or other immunostimmulating disease. For example, Kaplan et al (Am. J. Hematol., (2014), 89, 882-888) showed that AL and MM patients display an abnormally increased dimerisation of monoclonal FLC, accompanied by higher clonality values of FLC dimers than those of monomers. These abnormalities of FLC patterns were not observed in patients with MGUS, SMM, AL amyloidosis, and healthy individuals.

The significance of the polymeric variation of the FLC molecules was taken into consideration by the developers of the first sFLC assay (Bradwell et al, Clin. Chem., (2001), 47, 673-680). However, the published data shows that the antibodies generated bound the dimeric forms with greater avidity than the monomeric forms. It is therefore to be expected that the immunoassays generated using the mammalian derived polyclonal antibodies described by Bradwell et al will preferentially recognise the dimeric forms of the FLC.

It has been shown that both the Freelite and N-Latex assays considerably overestimate the concentration of monoclonal FLC, especially in samples with high titers, and that FLC polymerisation has been shown to be the cause (de Kat Angelino C M et.al 2010. Clin Chem. 2010 July; 56(7):1188-90., and Di Noto G et.al., Ann Clin Biochem. 2015 May; 52(3):327-36).

Currently there is no internationally accepted FLC reference material, nor a reference method. Significant differences are observed in the methodology of the current commercially available FLC-assays as well as in their responses to samples with varying multimeric forms of FLC.

Strategies have been suggested to mitigate the quantification of multimeric FLCs using mammalian derived antibodies, including the use of reducing agents for the selective cleavage of inter-light (L-L) chain disulphide bridge (Jerry and Kunkel, J Immunol Nov. 1, 1972, 109 (5) 982-991) in the assay formulation (WO2017144896A1). However, such strategies require additional steps and/or add to the complexity of the assay and such methods have not been successfully applied as yet.

Being able to quantify FLCs with less dependence on the polymerisation/multimeric state of the sample is important. It would also be advantageous to provide an assay which specifically detects λ or κ FLC, with reduced dependence on the multimeric form. The FLC antibodies must recognise only epitopes that are “hidden” in intact immunoglobulins to avoid falsely elevated FLC results from cross-reaction with light chains in whole immunoglobulins.

The present inventors have found that immunoassay reagents based upon avian-derived antibodies (IgY) address the problems associated with the different multimeric forms of FLCs.

### SUMMARY OF THE INVENTION

In a first aspect there is provided a method for assaying FLCs in a mammalian sample comprising the use of at least one avian-derived antibody (IgY).

In a second aspect there is provided a method for screening, diagnosis, monitoring or prognosis of a disease in a patient comprising conducting an assay method as described in the first aspect and comparing a result of the assay with at least one pre-determined threshold value.

In a third aspect there is provided a use of avian-derived antibodies (IgY) in an immunoassay for assaying FLCs in a mammalian sample.

In a fourth aspect there is provided an assay kit comprising at least one avian-derived antibody for use in assaying FLCs in a mammalian sample.

Various embodiments of the invention are described herein and are applicable to all aspects of the invention, where technically viable. All embodiments described herein may be used alone or in combination with all other embodiments, where technically viable.

In one embodiment, the avian-derived antibodies are chicken antibodies.

In another embodiment, the sample is serum, plasma or urine.

In another embodiment, the FLCs are λ, κ or total FLCs.

In another embodiment, the disease is selected from smouldering multiple myeloma, intact immunoglobulin myeloma, light chain myeloma, non-secretory myeloma, monoclonal gammopathy of undetermined significance (MGUS), light-chain amyloidosis (AL amyloidosis), Waldenstrom's macroglobulinemia, Hodgkin's lymphoma, follicular centre cell lymphoma, chronic lymphocytic leukaemia, mantle cell lymphoma, pre-B cell leukaemia or acute lymphoblastic leukaemia

In some embodiments, the avian-derived antibody is attached to a support material.

Further features and advantages of the invention will become apparent from the following description, given by way of example only, and with reference to the accompanying figures.

### BRIEF DESCRIPTION OF THE FIGURES

FIG. 1. Shows a calibration curve for particles coated with the anti-kappa IgY.

FIG. 2. Shows a calibration curve for particles coated with the anti-lambda IgY

FIG. 3. Shows the effect of reducing agents on kappa antigen using SDS-PAGE

FIG. 4. Shows a chicken IgY anti-Kappa particle enhanced immunoturbidimetric response and the effect of monomeric vs dimeric forms

FIG. 5. Shows a Freelite anti-kappa response and effect of monomeric vs dimeric forms

FIG. 6. Shows the effect of reducing agents on lambda antigen using SDS-PAGE

FIG. 7. Shows a chicken IgY anti-lambda particle enhanced immunoturbidimetric response and the effect of monomeric vs dimeric forms

FIG. 8. Shows a Freelite anti-lambda response and effect of monomeric vs dimeric forms

### DETAILED DESCRIPTION

This invention relates to a method for assaying FLCs in a mammalian sample comprising the use of at least one avian-derived antibody (IgY). The avian-derived antibody will be an anti-FLC antibody and may be an anti-kappa FLC or anti-lambda FLC antibody. Measuring the amount of κ FLCs, λ FLCs, total FLCs and/or the ratio of κ FLCs to λ FLCs (κ FLCs:λ FLCs or κ:λ ratio), provides information relating to several different diseases or disease states in patients. Normal plasma cells produce excess light chains which are excreted into serum. Approximately twice as many kappa to lambda light chains are produced in healthy humans. In healthy individuals these excess FLCs are reabsorbed and metabolised in the kidney. Individuals with certain diseases have a higher concentration of FLC and/or an altered ratio of κ FLCs to λ FLCs. Monoclonal gammopathy is a disorder caused by abnormal proliferation of a single clone of plasma cells. Conventionally, an increase in one of the λ or κ FLCs is looked for. For example, multiple myelomas result from the monoclonal multiplication of a malignant plasma cell, resulting in an increase in a single type of cell producing a single type of immunoglobulin. This results in an increase in the amount of FLC, either λ or κ, observed within an individual.

The present inventors have surprisingly found that avian-derived antibodies may bind the FLCs with stronger affinity than mammalian antibodies and/or may measure the different multimeric forms more equally. It would be a particular advantage if different multimeric forms could be detected with substantially equal affinity as, for example, AL and MM patients display abnormally increased dimerisation of monoclonal FLCs (Kaplan et al, 2014). Existing sFLC assays use antibodies generated by immunising laboratory mammals. Without wishing to be bound by theory, it is thought that some of the issues in existing assays may be attributed to the short phylogenetic distance between humans and the mammals used for generating the antibodies.

Methods of measuring the amount of a particular target molecule in a sample using a binding molecule that specifically binds to the target are well known in the art. Any suitable immunoassay method may be used in appropriate aspects of the present invention.

In some embodiments, the assay is an in vitro assay. In some embodiments, the assay is a nephelometric, turbidimetric, flow cytometric, lateral flow, radial follow, immunofixation electrophoresis (IFE) or Enzyme-Linked Immunosorbent Assay (ELISA). In some cases, the assay is a turbidimetric assay. In some cases, the assay is an ELISA assay.

In some embodiments, the assay comprises the use of a biosensor comprising the avian-derived antibodies and a physicochemical transducer or detector that transforms one signal to another (e.g. optical, piezoelectric, electrochemical, electrochemiluminescence etc.) to measure and quantify. In particular, the biosensor may convert the binding event of the avian-derived antibody to its antigen (FLC) into a measureable signal (e.g. a change in optical properties), detect that signal and process that detected signal into an assay result.

In some embodiments, the assay is a multiplex assay. Multiplexing allows for simultaneous quantification of multiple analytes in one sample. In some cases the multiplex assay is a lateral flow device containing multiple test lines. In some cases the multiplex assay is a fluorescent assay. In some embodiments the multiplex assay uses fluorescent beads. In some cases, the multiplex assay comprises colour-coded beads which are pre-coated with analyte-specific capture antibodies. In some cases, the multiplex assay comprises colour-coded beads which are pre-coated with analyte-specific capture antibodies, for example, a Luminex xMAP assay.

In some embodiments, the assay is laboratory based. In some cases, the assay is a point of care assay.

As used herein, an “assay” or “assay method” may be a qualitative or quantitative assay method. For a qualitative assay, the assay may return a binary result (e.g. positive or negative) indicating that the FLC concentration is above or below a pre-determined threshold. Such assays are particularly useful in initial screening to see whether further, more detailed analysis, may be justified. A quantitative assay will generally return a numerical result on either an absolute or an arbitrary scale. Such assays are valuable for FLC measurement because changes in FLC concentration may indicate the severity or progression of a disease, such as in the case of “smouldering” multiple myeloma, where regular testing may be provided. In such conditions, a stable numerical value for the FLC assay may indicate a stable disease and/or disease in remission. In contrast, a changing value may indicate escalating disease. For example, increasing total FLC concentration or increasingly abnormal kappa:lambda ratio may indicate progressing disease. Similarly, decreasing total FLC or more normal kappa:lambda ratio may indicate successful treatment and/or management.

In some embodiments, one or more of the IgY used in the assay may be labelled (e.g. to allow for detection of binding of the target molecule to the binding molecule). In some cases, the IgY is labelled by fluorescence, luminescence, radioactivity, isotopic labelling or conjugation to an enzyme, binder, particle, or substrate. In some cases, the IgY is labelled with an enzyme capable of converting a substrate into a detectable analyte. Such enzymes include horseradish peroxidase, alkaline phosphatase and other enzymes known in the art. In some cases, the IgY is conjugated to one member of a binding pair such as a specific antigen or biotin.

In some embodiments, the IgY is immobilised to a particle or surface. Immobilisation allows for processes such as separation of the immobilised IgY (and any bound component(s)) from the fluid phase. Such particles or surfaces may be any size and any material which can readily be separated from the fluid phase (e.g. 10 nm to 10 cm or 100 nm to 1 cm) and can include surfaces or larger items such as regions of glass or plastic surfaces or wells in microtiter plates (e.g. 96, 384 or 1536 well plates). Attachment to particles can also be used as a labelling method since agglutination of small particles can increase scattering and thus the turbidity of a sample.

In some cases, the IgYs are coated on nanoparticles having a mean diameter of at least 40 nm (e.g. 40 to 300 nm), such as 50 to 260 nm or preferably 80 to 200 nm. Suitable materials for particles and surfaces to which IgY may be attached include inorganic materials such as glass, ceramics, metals or metal oxides (e.g. silica, titania, zirconia); synthetic polymers such as thermoplastic or thermosetting polymers (e.g. polyolefins, polystyrenes, polyesters, polyamides, polycarbonates, polyurethanes, epoxy resins or phenolic resins); and/or natural polymers or modified (semi synthetic) natural polymers (e.g. latex rubber, cellulose, starch, protein polymers such as silk).

One particular example is attachment of the IgYs to nanoparticles made of glass, silica, latex, metal (e.g. gold) or a polymeric material (e.g. polyethylene or polystyrene).

One particular example is attachment of the IgYs to nanoparticles of latex particles (Ikerlat Polymers S.L., Spain) with said method comprising:
providing an immunoparticle according to any embodiments thereof;
reacting the sample with said immunoparticles;
detecting a change in reflectance, scattering or transmittance of the sample, wherein said change in reflectance, scattering or transmittance of the sample is indicative for the amount of FLC in the sample.

The sample is a sample chosen from blood plasma and blood serum and the change in reflectance, scattering or transmittance is a change in transmittance and the method is a turbidimetric method.

According to an embodiment of the above and the change is a change in reflectance or scattering and the method is a nephelometric method.

It is a significant advantage that the method can be performed as a turbidimetric or nephelometric method, as such methods can be automated, and performed on existing clinical analysers, that are in routine use in most clinical chemistry laboratory globally.

In certain embodiments, the IgY specific binder may be capable of specifically binding λ (lambda) or κ (kappa) FLC. Specific binding in this context may be taken has having less than 5% (e.g. 0.001% to 5%), preferably less than 1% cross-reactivity. That is to say, a lambda-specific IgY may have a cross-reactivity for kappa of less than 5% or less than 1%. Correspondingly, a kappa-specific IgY may have a cross-reactivity for lambda of less than 5% or less than 1%.

Cross-reactivity is the relative signal generated in an assay method between use of the target antigen in the assayed sample and the corresponding assay run with an alternative antigen in the sample at the same concentration. In the present case, cross-reactivity of a lambda-specific IgY for kappa will be the relative signal generated in an assay method when using a kappa FLC containing sample, relative to signal from the same assay method conducted on a lambda FLC containing sample. Correspondingly, the cross-reactivity of a kappa-specific IgY for lambda will be the relative signal generated in an assay method when using a lambda FLC containing sample, relative to signal from the same assay method conducted on a kappa FLC containing sample.

In some embodiments, the FLCs are selected from the group consisting of kappa FLCs, lambda FLCs, and mixtures thereof.

In some embodiments, the sample is a sample of blood, serum, plasma, saliva, urine, cerebrospinal fluid or other biological fluid. The sample is preferably a sample of serum, plasma or urine. The test sample is obtained from a mammal, preferably a human.

The avian-derived antibody (IgY) exhibits affinity for the constant domain of at least one mammalian immunoglobulin light chain. According to embodiments thereof, said IgY polyclonal antibodies binds specifically to the lambda or kappa FLC antigen with a KD of less than 20.0E-08 M (e.g. 20.0E-08M to 1.0E-12M), preferably 10.0E-09 M, and most preferably less than 2.0E-09 M. A suitable approach to measure affinity of binding of an antibody to its antigen is by surface plasmon resonance (SPR) using e.g. the Biacore™ assay platform and software (GE Healthcare).

In some embodiments, the avian-derived antibody is an antibody from an avian species selected from chickens, ducks, geese, turkeys or quail. In a preferred embodiment, the avian-derived antibodies in all aspects and embodiments herein may be chicken antibodies. The chicken antibodies can be polyclonal, monoclonal, or recombinant monoclonal antibodies. Monoclonal, or recombinant monoclonal antibodies may be used individually or in combination (e.g. as a multi-clonal preparation).

The antibodies of the invention may be purified from eggs from immunized chickens, immunized with native monoclonal, or polyclonal FLC's or recombinant antigens specific to the constant regions of immunoglobulin light chains kappa and lambda.

The antibodies of the invention may be purified using well-known antibody purification techniques. Suitable examples of antibody purification technology that may be used to purify antibodies of the invention comprise precipitation (salting-out) with ammonium sulphate or the like, ion exchange chromatography using a diethylamino-ester (DEAE) derivative, a carboxymethyl (CM) derivative, or the like, hydroxyapatite chromatography, gel filtration chromatography, and affinity chromatography using Protein A or Protein G, among others, including binding to antigen against which the antibody has been raised. It will be appreciated that combinations of the techniques suggested above may be utilised in purification of the antibody.

In some embodiments, the IgY is capable of specifically binding λ or κ FLCs. By “specifically binding” it is meant that the avian-derived antibody does not significantly bind to any molecules other than the desired FLC. For example, the anti-lambda antibody recognises the kappa antigen or the light-chain of whole molecule immunoglobulins only to a much lesser extent than the lambda FLC. This cross-reactivity can be used to generate a relative signal comparing binding of the target antigen vs an alternative antigen. In some cases, the cross reactivity is less than 1%.

In some embodiments, the IgY has substantially equal reactivity with monomeric and dimeric forms of FLC. This indicates that a substantially equal immunoassay signal is generated from a certain concentration of FLC in a sample, irrespective of the polymeric nature of the FLC antigen. In some cases, the IgY has substantially equal reactivity with all multimeric forms of FLC.

As used herein the term “substantially equal” indicates that two values differ by less than 20%, preferably less than 10% and more preferably by less than 5%

In some embodiments, at least two IgYs with different specificity are used. In some cases where there are at least two IgYs, one IgY has higher affinity for either λ FLC of κ FLC and the other has substantially the same affinity for λ FLC and κ FLC.

In some embodiments, the ratio of κ FLCs:λ FLCs in the sample is determined. In some cases the ratio of κ FLCs:λ FLCs is compared to a normal range for the ratio of κ FLCs:λ FLCs, and any significant deviation is taken as indicating the existence of a disease state and/or a change in the activity or severity of a disease state. A specific ratio or deviation of that ratio above or below a threshold value may be attributed to a specific disease or to a group of diseases which may be disambiguated by further tests. The change in kappa to lambda ratio over time may also indicate a change in the severity or progression of disease (e.g. indicating relapse or remission)

Normal ranges for the ratio of kappa to lambda light chains have been established previously (e.g. Rajkumar et al. Blood (2005) 106 (3): 812-817). These normal ranges were established using a single manufacturers assay (the Freelite™ Assay, The Binding Site Ltd, Birmingham, UK), and might not be the ranges identified in other FLC assays.

In one embodiment, an abnormal FLC ratio may be defined as a kappa to lambda chain ratio of less than 0.26 or more than 1.65, for example a ratio of less than 0.10 or more than 5.0. In certain embodiments, an increasing risk of disease progression (e.g. to active myeloma from precursor conditions such as MGUS, smouldering myeloma or solitary plasmacytoma of the bone) may be associated with increasing deviation from normal kappa:lambda ratio. Similarly, worsening prognosis in certain proliferative conditions (e.g. multiple myeloma or chronic lymphocytic leukaemia) may be associated with increasing deviation from normal kappa:lambda ratio.

In some embodiments, the amount of total FLC concentration is measured. In some cases, the total FLC concentration is compared to a standard, predetermined value to determine whether the total amount of FLC is higher or lower than a normal value. A normal range for kappa and lambda chain concentration in the serum of healthy individuals has been defined as 3.3-19.4 mg/L for kappa and 5.7-26.6 mg/L for the lambda chains [Katzmann et al. 2002, Clin Chem 48: 1437-1444]. These ranges were defined using the the Freelite™ Assay, (The Binding Site Ltd, Birmingham, UK), and might not be the ranges identified in other FLC assays. Calibration of the FLC assays in use today are fraught with difficulties in standardisation. Currently there is no internationally accepted reference measurement system for sFLC (Tate et al. 2009. The Clinical biochemist. Reviews vol. 30, 3: 131-40), therefore different FLC assays may result in markedly discrepant results (Schieferdecker et al. 2020. Blood cancer journal vol. 10, 1 2. 9), with an average overestimation of the FLC concentration of the Freelite assay being estimated to 10-fold overestimation (Helden etal. 2019. Hematol Med Oncol 4:1-7). In this case, the actual normal range for kappa and lambda chain concentration in the serum of healthy individuals may be as low as 0.33-1.94 mg/L for kappa and 0.57-2.66 mg/L for the lambda chains.

Suitable cut-off values for greater than normal FLC concentration may depend upon the context of the assay—broad first screening assays, for example, may use a low FLC concentration as “normal” and accept a higher proportion of false positives, which will be discounted after further investigation. Assays used as a factor in deciding upon aggressive or invasive tests or treatment may utilise a higher value for “normal”. In certain embodiments, therefore, normal FLC concentrations for kappa FLC may be considered to be 0.2 to 50 mg/L, such as 0.2 to 5 0 mg/L, 0.33-1.94 mg/L, 2.0 to 50 mg/L or 3.3 to 19.4 mg/L. A significant deviation from the normal range may be considered to be, for example, anything above the normal range, or 10% above the upper end of that range or, for example, 20% above the upper end of that range, depending upon the clinical context.

Higher than normal concentrations of sFLC are associated with a significant increase in the likelihood of reduced survival. The total FLC concentration may also be monitored in a subject over time with increasing total FLC concentration indicating increasing severity of disease (e.g. relapse) and decreasing total FLC concentration indicating reduced severity of disease (e.g. remission).

Being able to measure both the total sFLC concentration and ratio of κ FLCs:λ FLCs is advantageous as for patients with impaired renal clearance or polyclonal increases in immunoglobulins, sFLC levels may be elevated, but the sFLC κ:λ ratio will be normal. Whereas, for patients with an abnormal expansion of either a kappa- or lambda-producing plasma cell clone, the sFLC κ:λ ratio usually becomes abnormal.

Abnormal free kappa/lambda ratio is associated with the increased risk of progression to active myeloma from precursor conditions such as MGUS, smouldering myeloma, solitary plasmacytoma of the bone, and is prognostic of a worse outcome in multiple myeloma and chronic lymphocytic leukaemia. The assays of the present invention may thus be used as an aid in determining the prognosis or appropriate treatment regime in any such conditions.

In an additional aspect, the invention therefore provides for a method of distinguishing a monoclonal plasma cell disease (such as monoclonal gammopathy and/or monoclonal myeloma) from renal impairment and/or a polyclonal plasma cell disease, the method comprising measuring the ratio of κ FLCs:λ FLCs in a patient having elevated total FLCs where an abnormal ratio of κ FLCs:λ FLCs indicates a monoclonal plasma cell disease.

Also provided is a method for screening, diagnosis, monitoring or prognosis of a disease in a patient comprising a method for assaying FLCs in a mammalian sample comprising the use of at least one avian-derived antibody (IgY) and comparing a result of the assay with at least one pre-determined threshold value.

In some embodiments, the disease is a B-cell associated disease. In some cases, the disease is selected from smouldering multiple myeloma, intact immunoglobulin myeloma, light chain myeloma, non-secretory myeloma, monoclonal gammopathy of undetermined significance (MGUS), light-chain amyloidosis (AL amyloidosis), Waldenstrom's macroglobulinemia, Hodgkin's lymphoma, follicular centre cell lymphoma, chronic lymphocytic leukaemia, mantle cell lymphoma, pre-B cell leukaemia or acute lymphoblastic leukaemia.

In some embodiments, after diagnosing the subject as having a disorder, the method further includes administering to the subject a therapeutic agent to treat the disorder (e.g. a therapeutically effective amount).

In some embodiments, after diagnosing the subject as having a disorder, the method further includes performing a treatment such as a plasma exchange or a stem cell transplant.

Also provided is a use of avian-derived antibodies (IgYs) in an immunoassay for assaying FLCs in a mammalian sample.

Also provided is a use of at least one avian-derived antibody (IgY) in the manufacture of an assay kit for assaying FLCs in a mammalian sample.

Also provided is an assay kit comprising at least one avian-derived antibody (IgY) for use in the assay of FLCs in a mammalian sample.

In some embodiments, the assay kit further comprises at least one FLC reference sample.

In some embodiments, the IgY in the assay kit is attached to a substrate.

The assay kit can quantify the amount of λ or κ FLC or the total amount of FLC in the sample.

Also provided is a method of distinguishing between at least two B-cell associated diseases in a mammalian subject.

In a further embodiment, the present invention relates to a method for assaying Free Light Chains (FLCs) in a mammalian sample comprising the use of at least one avian-derived antibody (IgY) wherein the FLC monomer:dimer ratio is determined. This determination may be achieved by comparison of the method of the present invention with alternative methods which respond differently to FLC monomers and FLC dimers. In particular, since the assay method of the present invention provides results which are comparatively insensitive to the monomer:dimer ratio (see Examples below) and certain other known assay methods provide greater sensitivity for certain multimeric forms, a comparison of the results of two assay methods conducted on the same sample may provide information on the multimeric composition of the FLC content of that sample. Thus, by determining the deviation of the results between the assay method of the present invention and an alternative assay method (such as the Freelite or N-latex assay methods), the monomer:dimer ratio may be determined. This applies particularly where the alternative assay is based on mammalian (e.g. sheep, mouse or rat) antibody binding.

In one embodiment, the method of the present invention may thus comprise additionally conducting an alternative FLC assay on the same sample using an alternative assay method and comparing the results of that alternative assay method with those of the assay method comprising the use of at least one avian derived antibody (as described in any embodiment herein). Such alternative assay method may be, for example, an assay method based on binding of mammalian antibodies, such as sheep antibodies. Such mammalian antibodies will be anti-FLC antibodies and may be an anti-kappa FLC or anti-lambda FLC antibodies. Such mammalian antibodies May be monoclonal antibodies (e.g. multiple monoclonal antibodies) or polyclonal antibodies.

In one embodiment, the alternative assay method may be a turbidimetric, nephelometric or electrophoretic assay method.

In one embodiment, both of the assay method comprising the use of at least one avian derived antibody (as described in any embodiment herein) and the alternative assay method (e.g. comprising the use of mammalian antibodies) utilise the same detection method (such as turbidimetry, nephelometry or fluorescence).

The invention will now be illustrated by reference to the following non-limiting examples:

### EXAMPLES

### Example 1: Preparation of Polyclonal Avian Antibodies and Affinity Purification Thereof

### a) Preparation of Avian FLC Antibody.

The following immunization protocol may be used for the generation of polyclonal IgY antibodies against human FLC's:

Ten to twenty chickens are used for each immunization experiment. 10 mg of highly purified immunogen in phosphate buffer was emulsified with Freund's adjuvant and injected intramuscularly or subcutaneously into the chickens. The injection was repeated every 2 to 4 weeks. 10 to 12 weeks after the start of the injections, eggs were collected. The egg yolk was isolated from the eggs, and the IgY fraction from the egg yolk was delipidated then isolated by ammonium sulphate precipitation in a conventional manner according to prior art methods of egg antibody isolation (reviewed by Larsson A, et.al 1993 Poultry Science 72:1807-1812, 1993).

The immunogen in the example above is selected from purified Bence Jones proteins, polyclonal free light chains purified from patients suffering from acute kidney failure, purified FLC's generated from the fractionation of whole molecule immunoglobulins, or recombinant peptides corresponding to the amino acid sequences of the constant regions of immunoglobulin light chains.

### b) Affinity Purification of Polyclonal Antibody Fraction.

### Absorption Step:

10 mg of purified IgG, IgA, and IgM proteins were immobilised on a HITRAP NHS-activated HP column from GE Healthcare in accordance with the method described in the package insert of the column.

The isolated IgY fraction was diluted to 4 mg/ml in phosphate buffered saline. 200 to 300 ml of this IgY solution was passed through the column, and the flow-through fraction collected.

This flow-through fraction was then then affinity purified against mixtures of the respective FLCs using the following method:
5 mg of highly purified human free light chains was immobilized on a HITRAP NHS-activated HP column from GE Healthcare in accordance with the method described in the package insert of the column.
100 ml of the flow-through fraction was passed through the column, followed by 25 ml phosphate buffered saline.
The antibodies with specific affinity for the immobilized FLC's was eluted from the column with 15 ml of 0.1 molar citrate buffer pH=2.9. The eluted specific anti-FLC antibodies were dialyzed against phosphate buffered saline and concentrated to 3 to 5 mg/ml using an Amicon Ultra centrifugation filtration device with molecular weight cut-off of 30.000 Dalton.

### Example 2: Turbidimetric Assay for FLC's

### (a) Preparation of Anti-FLC Antibody Coated Nanoparticles

An Aliquot (e.g. 5 mg IgY) of the anti-kappa or anti-lambda antibodies prepared in Example 1, was dialysed against a MOPS buffer pH 8.4-8.6, before adding a 1 ml aliquot of 4% w/v chloromethyl activated nanoparticles (available from the likes of Ikerlat Polymers S.L., Spain or Thermo Fisher Scientific Inc. USA).

Following addition of the nanoparticles to the purified anti-FLC antibodies the mixture is agitated for 24-72 hours at 33 degrees Celsius. An equal volume of Glycine blocking buffer, pH 8.6, containing 10 mg/ml bovine serum albumin in boric buffer was added, and the mixture incubated at 33 degrees Celsius for 4-18 hours

Following this incubation, the particles are then diluted to total volume of 10 ml and dialysed, using a Float-A-Lyzer G2 dialysis device with a molecular weight cut-off of 1.000 kDalton, against 2, 2000 ml volumes of a Tris-NaCl buffer at pH 8.8 containing 0.1% Tween® 20, 10 mg/ml egg albumin and preservatives before adjusting to the final particle concentration in this storage buffer (R2) using dilution or centrifuge concentration.

### (b) Assay for FLC's Using Anti-FLC Antibody-Coated Nanoparticles

Calibrators for the assay were prepared using purified FLC's and the values assigned using the OD280 of the purified FLC's. The calibrator range was 0-1.6-3.1-6.3-12.5-25.0-50.0 mg/L.

An assay buffer (R1) was prepared by mixing soluble polymers like polyethylene glycol, salts and pH buffers, and the formulation was optimised until the turbidimetric signal of the assay results in a calibration of the assay corresponded to the Binding Site Freelite assay calibration. An example of the assay buffer is 25 mM TRIS, 180 mM NaCl, 0.1% Tween® 20 and 0.4% PEG 6000 at pH 7.4.

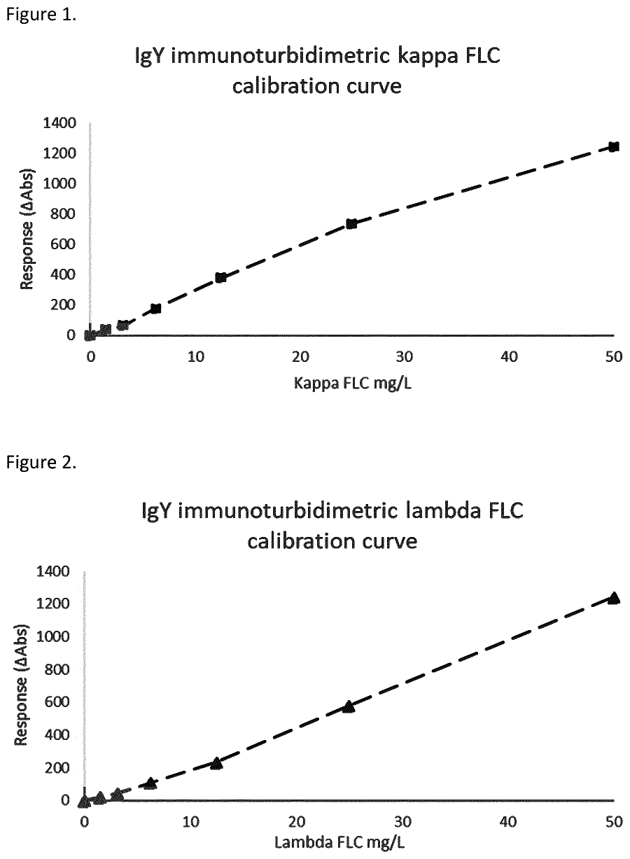

The latex enhanced turbidimetric immunoassay was established on a clinical chemistry analyser (ALCOR, Edif Instruments s.r.l. Via Ardeatina, 132-00179 Roma). The typical FLC assay used 3 μl of sample, 180 μl of R1 (assay buffer) and 60 μl R2 (antibody-coated Nanoparticles) Calibration curves are shown in FIG. 1 for the anti-kappa nanoparticle and in FIG. 2 for the anti-lambda nanoparticle.

### Example 3: Study of Monomer and Dimer Structure for Kappa and Lambda FLCs

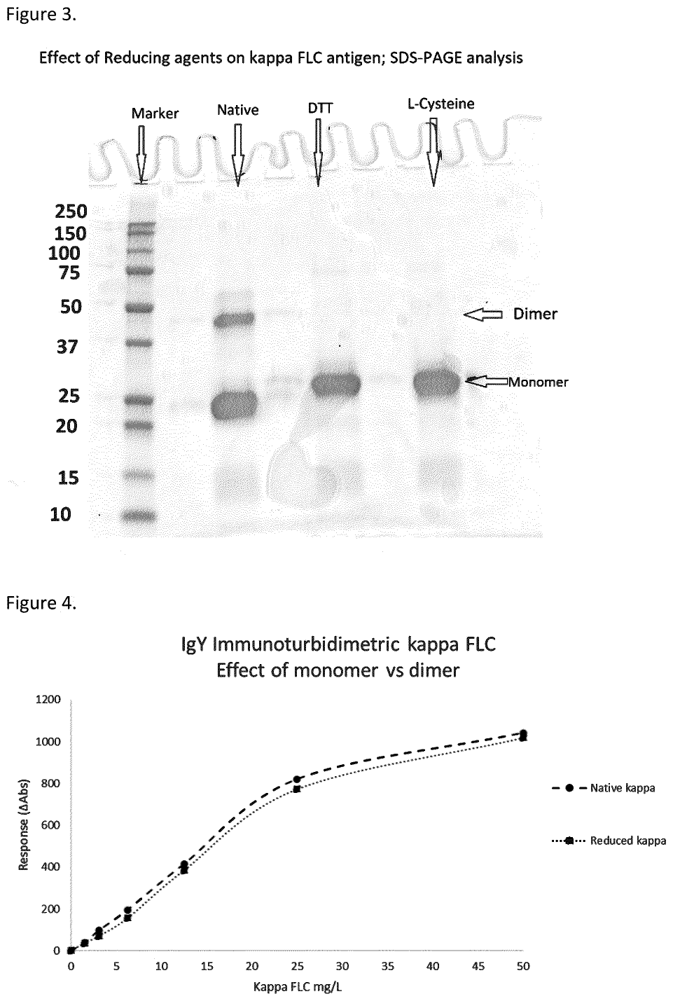

FIG. 3 shows an SDS-PAGE gel of kappa FLC antigen in native and reduced form. A purified kappa antigen was run on an SDS-PAGE (5-20%) gel. The lane marked native contains the non-reduced kappa antigen that clearly shows the monomeric kappa FLC at approximately 24 kDa, and the dimeric kappa FLC at approximately 48 kDa. The band corresponding to the monomer is more intense than the band for the dimer. The lane marked DTT was the same kappa antigen that had been reduced with 100 mM DTT prior to the running on the SDS_PAGE. The lane marked L-cysteine was the same kappa antigen that had been reduced with 15 mM L-cysteine before running on the SDS-PAGE gel. It is clear that both the reducing agents (DTT and L-cysteine) efficiently convert the dimeric kappa FLC into the monomeric form of the kappa FLC since no band corresponding to the MW of the dimer is visible.

FIG. 6 shows an SDS-PAGE gel of lambda FLC antigen in native and reduced form. A purified lambda antigen was run on an SDS-PAGE (5-20%) gel. The lane marked native contains the non-reduced lambda antigen that clearly shows the monomeric lambda FLC at approximately 24 kDa, and the dimeric lambda FLC at approximately 48 kDa. The band corresponding to the monomer is just visible while the band for the dimer is much more intense. The lane marked DTT was the same lambda antigen that had been reduced with 100 mM DTT prior to the running on the SDS_PAGE. The lane marked L-cysteine was the same lambda antigen that had been reduced with 15 mM L-cysteine before running on the SDS-PAGE gel. It is clear that both the reducing agents (DTT and L-cysteine) efficiently convert the dimeric lambda FLC into the monomeric form of the lambda FLC since little or no band corresponding to the MW of the dimer is visible.

### Example 4: Effect of Polymeric Structure of the FLC's in the Turbidimetric Assay

### (a) Conversion of Light Chain Dimers to Monomers

It is considered that kappa's FLC occur mainly as monomers, whilst the lambda FLC's mainly occur as dimers. A non-reducing SDS-PAGE gel confirmed that the kappa antigens used in the assay calibration consisted mainly as monomers (FIG. 3), whereas the lambda antigens consisted mainly as dimers (FIG. 6) in the native state.

Reducing agents such as glutathione, L-cysteine, tris(2-carboxyethyl)phosphine, B-mercaptoethanol, dithiothreitol (DTT) and dithioerythritol (DTE) reduce disulphide bonds between molecules, for example multimeric light chains. The efficiency of the reduction of the kappa dimers to kappa monomers by DTT and l-cysteine is shown in FIG. 3. The efficiency of the reduction of the lambda dimers to lambda monomers by DTT and l-cysteine is shown in FIG. 6.

### (b) Effect of the Polymeric Structure of the FLC's on the Chicken Antibody Based Turbidimetric Assay for FLC's

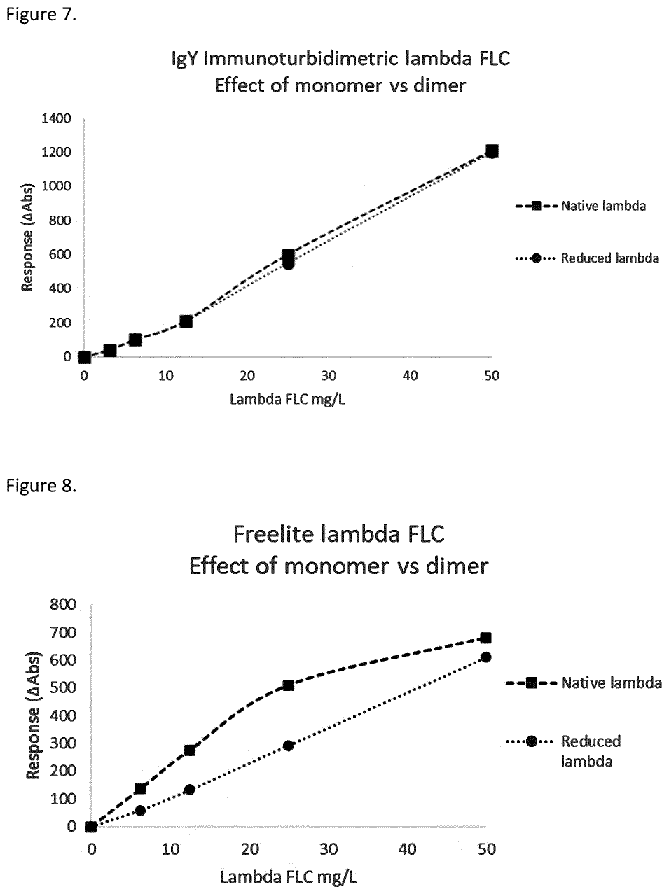

Two separate kappa and lambda calibrators series were prepared as before. To prepare the monomeric calibrator range a first set of calibrators was incubated with 15 mM l-cysteine in the assay buffer. The second set of calibrators (native calibrators containing dimers and monomer forms of the FLC) was incubated in the assay buffer without l-cysteine before generating standard curves with the immunoturbidimetric assay. The ALCOR clinical chemistry assay was used as described in Example 2. The assay calibration curves for the native, and monomeric kappa FLC calibrators are shown in FIG. 4. The assay calibration curves for the native, and monomeric lambda FLC calibrators are shown in FIG. 7.

### (c) Effect of the Polymeric Structure of the FLC's on the Freelite Kappa and Lambda Kits

The Freelite Human Kappa Free kit (product code LK016.CB) and Freelite Human Lambda Free kit (product code LK018.CB), both manufactured by the Binding Site, Birmingham, B15 1QT, UK, were configured to run on the ALCOR clinical chemistry analyzer. A person skilled in the art can optimize the parameter settings for these assays on any suitable automated clinical chemistry analyzer.

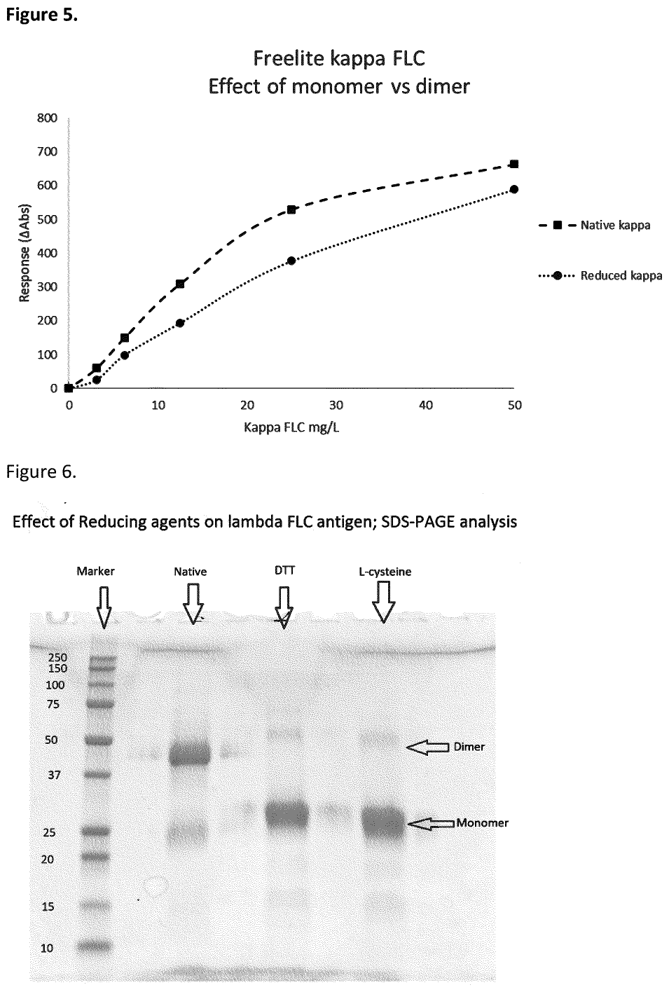

Two separate kappa and lambda calibrators series were prepared as before. To prepare the monomeric calibrator range a first set of calibrators was incubated with 15 mM l-cysteine in the assay buffer. The second set of calibrators (native calibrators containing dimers and monomer forms of the FLC) was incubated in the assay buffer without l-cysteine before generating standard curves with the Freelite assays. The assay calibration curves for the native, and monomeric kappa FLC calibrators are shown in FIG. 5. The assay calibration curves for the native, and monomeric lambda FLC calibrators are shown in FIG. 8.

1. A method for assaying Free Light Chains (FLCs) in a mammalian sample comprising the use of at least one avian-derived antibody (IgY).

2. The method of claim 1 wherein the IgY exhibits affinity for the constant domain of at least one mammalian immunoglobulin light chain.

3. The method of claim 1 or claim 2 wherein the IgY is labelled.

4. The method of claim 3 wherein the IgY is labelled by fluorescence, luminescence, radioactivity, isotopic labelling or conjugation to an enzyme, particle or substrate.

5. The method of claims 1 to 4 wherein the IgY is immobilised to a particle or surface.

6. The method of claim 5, comprising the use of a biosensor comprising an immobilised IgY as binding agent.

7. The method of any preceding claim wherein the assay is a nephelometric, turbidimetric, flow cytometric, lateral flow, immunofixation electrophoresis (IFE) or Enzyme-Linked Immunosorbent Assays (ELISA) assay.

8. The method of any preceding claim, wherein said assay is a turbidimetric assay.

9. The method of any preceding claim, wherein said IgYs are coated on nanoparticles having a mean diameter of at least 40 nm, such as 50 to 160 nm.

10. The method of any of claims 1 to 7, wherein said assay is an ELISA assay.

11. The method of any preceding claim, wherein said IgY specifically binds (or is capable of specifically binding) λ (lambda) or κ (kappa) FLC.

12. The method of any preceding claim, wherein the FLC is λ (lambda), κ (kappa) or total FLC.

13. The method of any preceding claim comprising the use of at least two IgYs with different specificity wherein one has higher affinity for lambda FLC and another has higher affinity for kappa FLC.

14. The method of any of claims 1 to 13 comprising the use of at least two IgYs with different specificity wherein one has higher affinity for either lambda FLC or kappa FLC and the other IgY has substantially the same affinity for lambda and kappa FLCs.

15. The method of any preceding claim, wherein the ratio of ratio of κ FLC to λ FLC (FLC κ:λ) in the sample is determined.

16. The method of claim 15, wherein the FLC κ:λ ratio is compared to a normal range for FLC κ:λ and any significant (e.g. >10% or >5%) deviation from the normal range calculated, with increasing deviation from the normal range being associated with increasing prospect of disease progression and/or worsening prognosis.

17. The method of any preceding claim, wherein said IgY has substantially equal reactivity with monomeric and dimeric forms of FLC.

18. The method of any preceding claim, wherein said IgY has substantially equal reactivity with all multimeric forms of FLCs.

19. The method of any preceding claim, wherein the mammalian sample is a sample of blood, serum, plasma, saliva, urine or cerebrospinal fluid or other biological fluid, preferably serum, plasma or urine.

20. A method for screening, diagnosis, monitoring or prognosis of a disease in a patient comprising conducting an assay method as claimed in any of claims 1 to 19 and comparing a result of the assay with at least one pre-determined threshold value and/or calculating a deviation of a result of the assay from a normal range.

21. The method of claim 20, wherein the disease is a B-cell associated disease.

22. The method of claim 21, wherein the disease is selected from smouldering multiple myeloma, intact immunoglobulin myeloma, light chain myeloma, non-secretory myeloma, monoclonal gammopathy of undetermined significance (MGUS), light-chain amyloidosis (AL amyloidosis), Waldenstrom's macroglobulinaemia, Hodgkin's lymphoma, follicular centre cell lymphoma, chronic lymphocytic leukaemia, mantle cell lymphoma, pre-B cell leukaemia or acute lymphoblastic leukaemia.

23. Use of avian-derived antibodies (IgYs) in an immunoassay for assaying FLCs in a mammalian sample.

24. Use as claimed in claim 1 in an assay method or any of claims 1 to 19.

25. Use of at least one avian-derived antibodies (IgY) in the manufacture of an assay kit for assaying FLCs in a mammalian sample.

26. An assay kit for use in a method according to any of claims 1 to 22.

27. An assay kit comprising at least one avian-derived antibody for use in the assay of FLCs in a mammalian sample.

28. The assay kit of claim 26 or 27, further comprising at least one FLC reference sample.

28. The assay kit of claim 26, 27 or 28, wherein the IgY is attached to a substrate.
